(19) United States
(12) Patent Application Publication
(10) Pub. No.: US 2022/0357066 A1
(43) Pub. Date: Nov. 10, 2022

(54) Ultraviolet Germicidal Irradiation Light and Bipolar Ionization Control by HVAC Thermostats

(71) Applicant: J. Drew, Inc. d/b/a Network Thermostat, Grapevine, TX (US)

(72) Inventor: Jerry DREW, Grapevine, TX (US)

(21) Appl. No.: 17/736,304

(22) Filed: May 4, 2022

Related U.S. Application Data
(60) Provisional application No. 63/183,998, filed on May 4, 2021.

(51) Int. Cl. F24F 11/64; F24F 8/22; F24F 8/30; G05B 19/042; A61L 9/20; A61L 9/22

(52) U.S. Cl. CPC F24F 11/64; F24F 8/22; F24F 8/30; G05B 19/042; A61L 9/20; A61L 9/22; F24F 2120/10

(57) ABSTRACT

An air sanitization and purification system controlled by a thermostat in wired or wireless communication with an ultraviolet germicidal irradiation light or bipolar ionization appliance, with operation of the light or appliance controlled according to a pre-defined operation schedule of a heating, ventilation and cooling (HVAC) system or by receipt of sensor data indicating the presence of a person with an enclosed area heated or cooled by the HVAC system.

### CLAIM OF PRIORITY

This application claims priority to U.S. Provisional Application No. 63/183,998 filed May 4, 2021 and is incorporated by reference herein in its entirety.

### TECHNICAL FIELD

This disclosure relates generally to an ultraviolet germicidal irradiation light (UV-C) and any of many other air purification products and technique such as Bipolar Ionization (BPI) used for air disinfection in connection with heating, ventilation and cooling (HVAC) thermostats, and specifically to a control for providing UV-C or other air purification techniques, including BPI to selectively and efficiently apply disinfection to areas on interest while eliminating application during periods in which areas of interest are vacant.

### BACKGROUND

The novel coronavirus COVID-19 pandemic of 2020 has caused massive illness, death and strife throughout the world. Among its many, many negatives COVID-19 has placed an enormous strain on the health care system and has caused change to life as we know it from business, nutrition, and recreation. Citizens have been encouraged to take drastic social distancing and hygiene measures like never before. Homes, businesses, health care facilities, arenas, schools and virtually every other public or private place at present and for the foreseeable future require various levels of sanitization efforts to attempt to stall or eliminate the spread of the virus. Virtually all in commerce and private life found themselves unprepared and often unable to adapt to the many demands caused by COVID-19.

Social distancing requirements require schools, for example, to offer remote learning. Those schools that resumed in person learning were required to reconfigure the layout of the classroom as well as require students and faculty to wear personal safety equipment, such as a mask. While the number of positive COVID-19 cases moves lower, going forward the need for heightened measures in anticipation of a possible “second strain” of COVID-19 or the next contagious disease is required. Proactive measures that commercial and residential structures can put in place to maintain a substantially cleaner air in their environment are ultraviolet germicidal irradiation and bipolar ionization. Ultraviolet germicidal irradiation is achieved through use of short wavelength ultraviolet UV-C light to kill or inactivate microorganisms by destroying nucleic acids and disrupting their DNA, leaving the organisms unable to perform vital cellular functions. Bi-polar ionization techniques split molecules in the air into positively and negatively charged ions, and reportedly help to remove viruses from the air and provide surface disinfection for surfaces within the area. Bipolar ions seek out atoms and molecules in the air and effectively neutralize particles containing bacteria, odors, and volatile organic compounds (VOCs) in the air.

UV-C light has been shown to be extremely effective in killing COVID-19 microorganisms, both on surfaces and airborne, along with other infectious organisms such as the common cold virus, the flu virus, fungus, spores, legionnaire bacteria and black mold. And like the flu virus, as Covid variants have become more evident, the particle sizes and basic structure have not changed. UV-C will continue to work effectively against the variants that Covid has presented. As such, portable UV-C light units have been used in locations such as hotel rooms and classrooms to kill any present COVID-19 microorganisms. In operation, the user places a light stand with the appropriate UV-C light installed within the room or area of interest. Due to the very harmful effects that UV-C light can cause including damage to the skin and eyes (even permanent damage or blindness), the user is required to set a timer or other delay type before and leave the room before the light is turned on to kill the harmful cells.

UV-C has been used for some time to clean HVAC evaporator coils and inhibit mold growth in HVAC systems. Direct exposure to UV-C can damage skin and eyes, and even cause temporary blindness. UV-C lamp installation in the ductwork of HVAC systems is common in hospital rooms where high air exchange rates draw the air across the lamps, cleaning the air. This same technique is now being used in standard room air applications, but there are no effective ways to determine if the lamp is working properly. Additionally, there are currently no known methods to log lamp run times or lamp efficiencies.

Typical UV-C lamp life is approximately 6,000-12,000 hours. For HVAC application, installation typically entails providing power to the lamp from a 120/240 VAC source at the HVAC equipment. The UV-C lamp is located across a point in the HVAC air circulation system where air will continuously pass. Exposure of the stream of air to the UV-C light rays result in killing any virus particles present in the air.

There are many drawbacks to killing virus microbes in this manner. The UV-C lamp in this application is left on 24 hours per day, leading to a required lamp replacement every 500 days for a 12,000-hour lamp. This method is less than ideal since most buildings are not used or occupied 24 hours per day. The majority of the lamp burn time, therefore, is typically wasted since the lamp is not cleaning air unless the air is moving across it.

To overcome this problem, another method has surfaced in which the lamp is turned on by interlocking the HVAC equipment fan with the power supply to the lamp, so it only burns when the fan is on, reducing the waste described above. Unfortunately, this causes a different issue with lamp life. UV-C lamps lose approximately one hour of lamp life every time they are turned on. This drastically reduces the effective life of the lamp because in regulating the temperature of a space, the HVAC unit continually is powered on and off, which in turn causes the UV-C lamp to be constantly turned on and off.

As an example, in a commercial environment standard HVAC equipment is turned on and off approximately three times per hour during an eight-hour period corresponding to a typical work day. Assuming a 221-day work year, the lamp life is reduced by about 5,300 hours (3 cycles/hr*8 hrs/day*221 days), giving it an effective life of only 6,700 hours in a single year (12,000−5300), assuming the high end of typical lamp life. This amounts to a forty-four percent reduction in effective life, without accounting for actual lamp runtime.

If the HVAC system typically runs 10 minutes per heating or cooling cycle, the lamp is burns for only four hours per day ((10 min*3 cycles/hr*8 hr per day)/60), which is not long enough to turn over room air to be an effective cleaning tool. This deems this method of air cleaning ineffective. Effective lamp life in this case does increase significantly though, from 1.37 years (500 days) in the first example, to 7.58 years in this example. 1,675 days (6,700 hours/4 hrs per day), and applying 221 workdays per year, results in an estimated lifetime of 7.58 years. Again, this is meaningless because the UV-C lamp does not operate long enough to be effective for cleaning.

In addition to wasted life span, there is also the basic unknown of whether the UV-C lamp is actually operating. This is a product of the inability to look at the lamp with the human eye because of damage to the eye caused by the light. In addition to the normal degraded lifetime of the light by the constant on and off status, this inability to monitor and replace the light immediately upon failure makes the general sanitization process all the more ineffective.

Overall, the issues with application of UV-C lighting to HVAC systems to serve as a sanitization means to combat COVID-19 and other infectious microbes are many. These include (1) maximizing effective lamp life via proper control; (2) knowledge of when the lamp needs to be replaced; (3) confirming that the lamp is operating; (4) alerting when the lamp is not operating; (5) alerting that the lamp requires replacement; (6) recording the lamp's operation (there may be more than one lamp needed in any particular HVAC system, depending on the supply plenum size and the air cfm); and (7) providing a simple method of selecting different UV-C lamp control profiles for different building use cases. These and other considerations are addressed and resolved by the presently described Ultraviolet germicidal irradiation (UVGI) light controller for HVAC systems. With this method, current and currently proposed (known at this time) local construction code and federal health requirements are met, without impacting outside air requirements or additional environmental impacts.

Similarly, bipolar ionization products are often also installed in HVAC equipment to achieve similar air and surface purification results as UV-C lights. And like UV-C lights, BPI appliances come with the same need for appropriate control during occupied hours and unoccupied hours of any facility, as their effective use requires airflow.

Bipolar ionization technologies also have similar issues. require regular maintenance and cleaning. These include (1) maximizing effective ionization needle life via proper control; (2) knowledge of when the unit needs to be replaced; (3) confirming that the unit is operating; (4) alerting when the unit is not operating; (5) alerting that the unit requires cleaning, (6) alerting when the unit requires replacement; (7) recording the unit's operation (there may be more than one unit needed in any particular HVAC system, depending on the supply plenum size and the air cfm); and (8) providing a simple method of selecting different UV-C lamp control profiles for different building use cases.

Both UV-C and BPI assemblies can also be used as free-standing units outside of HVAC equipment. As outside units they offer the same air disinfection and cleaning affects. With outside (in room) use the same control and alerting is needed.

### BRIEF DESCRIPTION OF THE DRAWINGS

The novel features believed characteristic of the invention are set forth in the appended claims. The invention itself, however, as well as a preferred mode of use, further objectives, and advantages thereof, will be best understood by reference to the following detailed description of illustrative embodiments when read in conjunction with the accompanying drawings and photographs, wherein:

FIG. 1A depicts a perspective view of a physical space in which an intelligent thermostat and UV-C lamp are used according to an embodiment of the present invention.

FIG. 1B depicts a perspective view of a physical space in which an intelligent thermostat and UV-C lamp are used according to an embodiment of the present invention.

FIG. 1C depicts a perspective view of a physical space in which an intelligent thermostat and an air purification appliance are used according to an embodiment of the present invention.

FIG. 2A depicts a detailed view of components and peripheral devices used in connection with an HVAC system equipped with an air purification appliance according to an embodiment of the present invention.

FIG. 2B depicts a detailed view of components and peripheral devices used in connection with an HVAC system equipped with an air purification appliance according to an embodiment of the present invention.

FIG. 3A depicts a detailed view of a standalone device associated with an air purification appliance according to an embodiment of the present invention.

FIG. 3B depicts a detailed view of a standalone device associated with an air purification appliance according to an embodiment of the present invention.

### DETAILED DESCRIPTION OF THE EMBODIMENTS

The detailed description set forth below is intended as a description of the present embodiments of the invention and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

Several embodiments of Applicant's invention will now be described with reference to the drawings. Unless otherwise noted, like elements will be identified by identical numbers throughout all figures. The invention illustratively disclosed herein suitably may be practiced in the absence of any element which is not specifically disclosed herein.

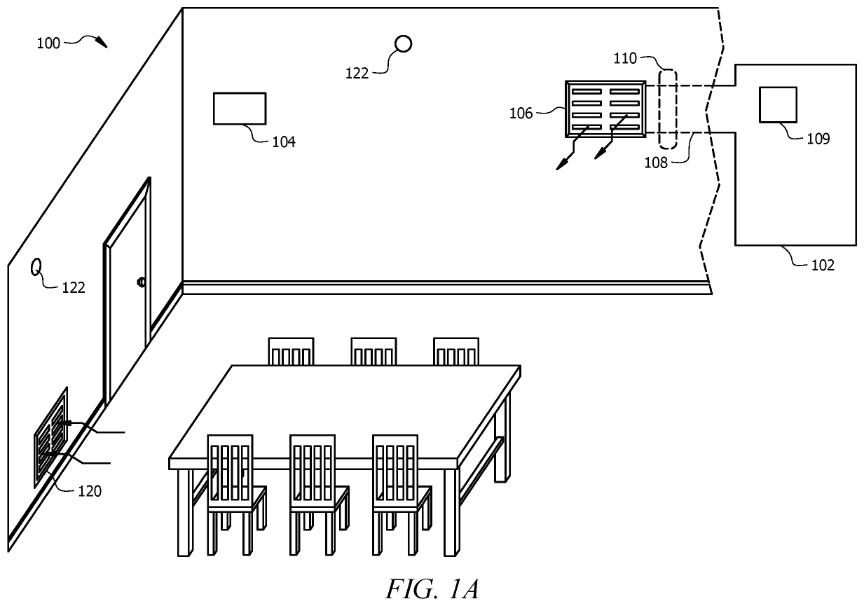

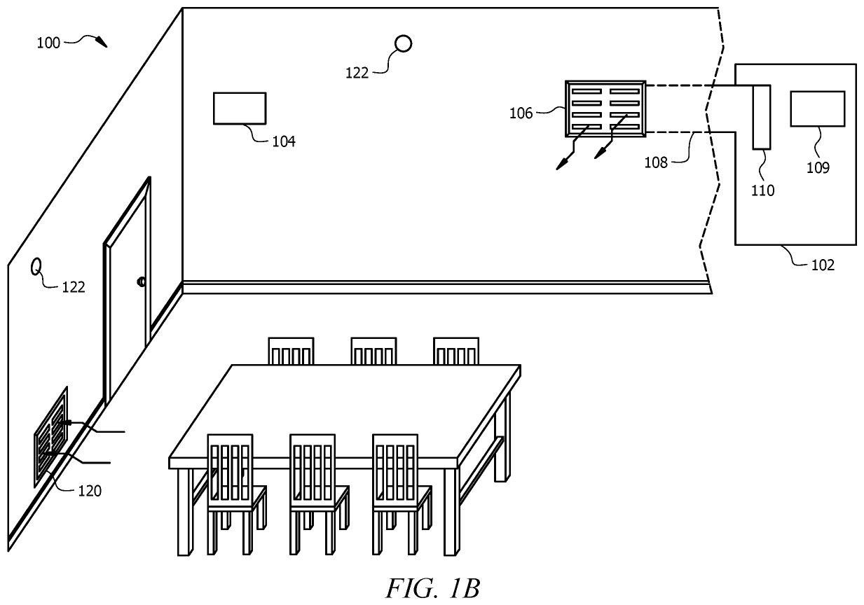

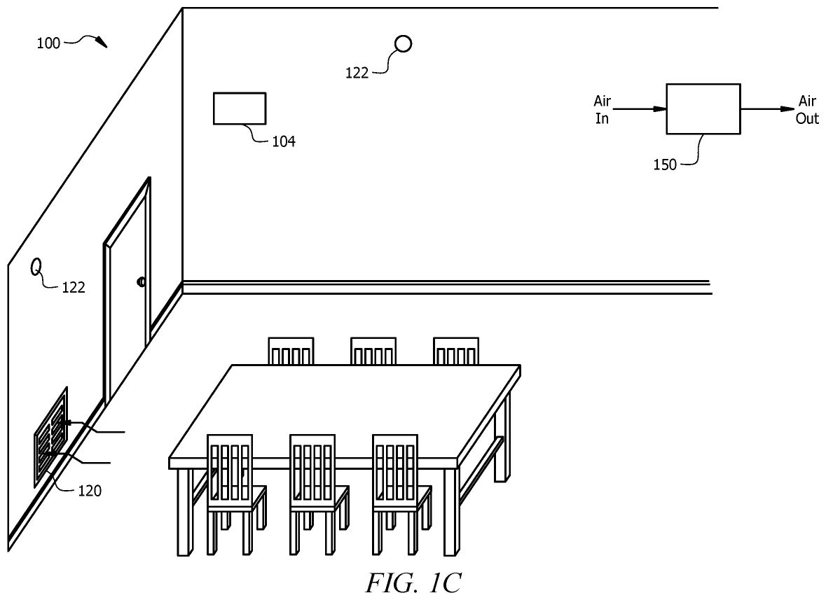

FIG. 1A depicts an environment in which an HVAC system equipped with an associated UV-C light to kill viral microbes such as COVID-19 operates. In FIG. 1A, room 100 is a common room in which contaminants are to be eliminated though operation of the present invention, such as a conference room, an office, or a classroom. As known, a school classroom or a conference room in a typical business office is occupied during weekdays and usually no longer than approximately eight hours per day. But throughout the business day, a conference room can be empty for much of the day, although it continues to be heated or cooled. An office is usually in heated and cooled through one of several HVAC system types including but not limited to rooftop units, split-systems, VRF, fan evaporator coil units, or its own large commercial HVAC plant that serves to heat or cool several classrooms, offices and other areas in parallel. For ease of illustration, however, in FIG. 1A, there is a local HVAC system 102 directly associated with room 100. This is by no means limiting, as HVAC system 102 can cool or heat several rooms, emit, and receive heated or cooled air through one vent or multiple vents and the like. In FIG. 1, again for ease of illustration, room 100 includes one or more vents 106 through which cooled or heated air enters room 100. Inlet vent 120 draws in air from room 100 to facilitate circulation of air into and out of HVAC unit 102.

In close proximity to vent 106 is UV-C lamp 110. In one embodiment, as shown in FIG. 1B, UV-C lamp 110 is situated in the interior of the HVAC unit in close proximity to where air enters HVAC duct 108. Preferably, UV-C lamp 110 is also situated in close proximity to the HVAC evaporator coil 109. Placement of UV-C lamp 110 next to coil 109 inside of the HVAC unit 102 allows for sanitizing the coil during the air filtration and sanitization process while the lamp is in use, killing the Covid microbes that are trapped within crevices of the coil. In this manner, all air passing through duct 108 and out of vent 106 and into room 100 is exposed to UV-C light waves. As a result, viral microbes are destroyed, or their growth impeded.

In one embodiment, lamp 110 is hard wired for AC power. Lamp 110 is also controlled via wired or wireless connection to intelligent thermostat 104. Intelligent thermostat 104 functions as a controller for lamp 110 and can also control various other items in the environment. For purposes of the presently described HVAC system having an associated lamp for destroying microbes, intelligent thermostat 104 includes an application specific integrated circuit, microcontroller(s), or a processor having an operating system enabling execution of application programs or code to control operation of UV-C lamp or BPI appliance, the associated HVAC unit and storing data associated with these operations. In one embodiment, the controller of the UV-C lamp or BPI appliance is embedded within intelligent thermostat 104. In an alternative embodiment, the controller of controller of the UV-C lamp or BPI appliance is external to the intelligent thermostat 104 but communicates with intelligent thermostat 104 by either wired or wireless communication.

In particular, intelligent thermostat 104 includes a user interface allowing users to input commands and control operation of the HVAC unit. The user interface takes many forms, including on the thermostat display, an embedded web page, a separate desktop application, a mobile application, or a cloud user interface. Among the controllable features are room temperature settings, daily and hourly heating and cooling scheduling, including on/off times, and UV-C lamp operational parameters. Control of the operations of UV-C lamp and associated methods disclosed herein can be performed by an embedded controller in a thermostat but also by a remote device in wired or wireless communication with the thermostat via a remote sensor bus. Note, however, that the control techniques described herein can also be housed in a standalone controller. Also, a standalone device that also communicates with other automation devices (home, commercial or industrial) with features that can be programmed to control operation of the UV-C lamp.

In another embodiment a controller acts as a master of the thermostat and the air purification appliances and/or the HVAC unit. A controller external to the thermostat comprises a processor and source code enabling the controller to operate the UV-C lamp and BPI appliance according to HVAC run schedule information received from the thermostat. In addition, the controller optionally receives

The UV-C lamp itself, along with its controller, can also be a standalone device for use in the situation in which retrofitting the HVAC system by installing the lamp inside of the HVAC unit is not option. A standalone UV-C unit according to an embodiment of the invention is shown in FIG. 1C. In FIG. 1C, standalone unit 150 provides microbe cleaning apart from the HVAC unit. Standalone unit 150 can be a tabletop unit, wall mounted, ceiling mounted, or a floor unit. The standalone unit 150 includes an air inlet and an air outlet to provide air flow through the unit. Once air passes through standalone unit 150 the UV-C or BPI capability within the unit performs sanitization of the air particles according to the techniques of that capability.

The embodiments described in FIGS. 1A, 1B and 1C above include a UV-C lamp 110 inside the HVAC system duct work in close proximity to vent 106 (FIG. 1A), inside the HVAC systems itself in close proximity to coils 109 (FIG. 1B) or within the room as a standalone unit 150 (FIG. 1C). In another embodiment, instead of an UV-C lamp serving in the capacities and locations described in FIGS. 1A, 1B and 1C, a BPI appliance is used in lieu of a UV-C lamp. Like the UV-C lamp, the BPI appliance is controlled by an embedded thermostat controller and is controlled in the same manner as described for the UV-C lamp.

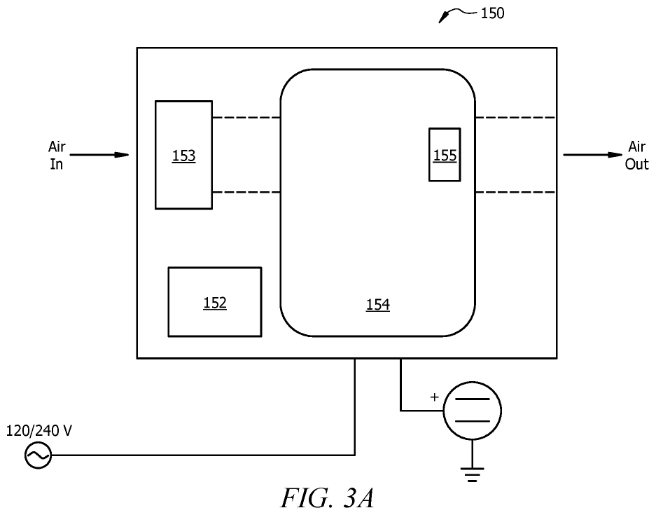

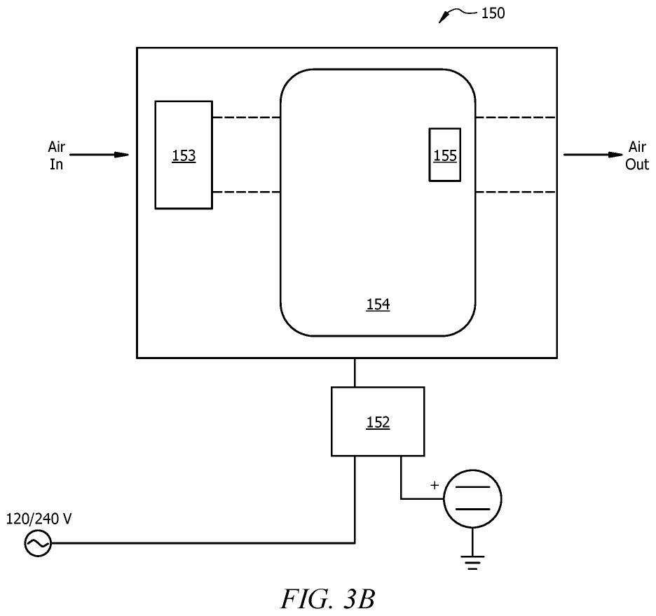

FIG. 3A provides a detailed view of standalone UV-C unit 150. In FIG. 3A, standalone UV-C unit 150 includes a fan 153 that draws air into unit 150 and in particular to cleaning chamber 154. Within cleaning chamber 154 is UV-C lamp 155 that works to kill contaminants, including Covid microbes as described above. Once lamp 155 cleans air entering cleaning chamber 154 the cleansed air leaves standalone UV-C unit 150 and enters the room. As standalone UV-C unit 150 continues to run, air within the room continues circulating and cleansing.

Also, in FIG. 3A, standalone unit controller 152 is shown. In this embodiment, standalone controller 152 is housed and powered by either A/C or D/C power within the standalone UV-C unit 150 which is powered by a connected A/C power source as shown. The standalone unit in this embodiment is not tied to nor communicates with intelligent thermostat 104. Standalone controller 152 provides the data collection and control capability described below with respect to the intelligent thermostat's 104 functionality and operation for intelligently controlling when the UV-C lamp 155 within standalone UV-C unit operates. Standalone controller 152 control a single UV-C unit or multiple UV-C units via wired or wired communication, regardless of whether standalone controller 152 is housed within the UV-C unit 150 case, as shown in FIG. 3A or outside of it as shown in FIG. 3B below. The various embodiments described herein, including the intelligent thermostat and remote sensor also control one or multiple UV-C lamps.

FIG. 3B depicts another embodiment of standalone UV-C unit 150, but with controller 152 located outside of the case of standalone UV-C unit 150 and is still powered by an A/C and or D/C power source. Locating the standalone UV-C controller 152 inside or outside of the case of standalone UV-C unit 150 results in the same intelligent control of UV-C lamp 155 and standalone UV-C unit 150. Standalone UV-C controller intelligently controls operation of fan 153 and UV-C lamp 155 in order to achieve maximum efficiency rather than simply setting on/off times for the light. Intelligent control is achieved, as discussed below, by virtue of collected data of the environment in which the standalone UV-C unit 150 sits, which results in scheduling UV-C lamp operation in the most efficient and cost-effective manner. Regardless of whether the described controller is a standalone (external) controller 152 or an embedded controller, operation of the UV-C lamp or BPI appliance is also controlled manually through the user interface of the standalone controller or through the intelligent thermostat if the controller is embedded in situations where the lamp or appliance needs to be turned off for maintenance or other situations, or turned on for testing, etc.

The standalone unit allows the user to place the lamp at a desired location for optimal results. The standalone device provides flexibility in terms of after-market installation of the UV-C lamp where installation within the HVAC unit itself is not available. Just like the thermostat unit with its embedded controller, the standalone unit is an intelligent unit in that it collects operation and environmental data and fine tunes operations in response to it. The standalone unit, like the thermostat unit, sends alerts to users concerning lamp life, performance, etc.

While the features, operation and control of the UV-C lamp is described in the context of an associated intelligent thermostat, the features and functionality herein described are also applicable to a controller of the described standalone device and control via a remote device in communication with the thermostat via the remote sensor bus. By disclosing the invention in the context of the controller residing in the intelligent thermostat is in no way limiting.

As discussed, the continued turning on and off UV-C lamp 110 throughout the day drastically reduced the effective life of the UV-C light. Moreover, many hours of heating or cooling a vacant space has the doubly detrimental impact of reducing the light's life by normal run hours as well as reducing light life by many unnecessary on-and-off events during the vacant hours. The controller function of thermostat 104, therefore, provide efficient and effective use of lamp 110.

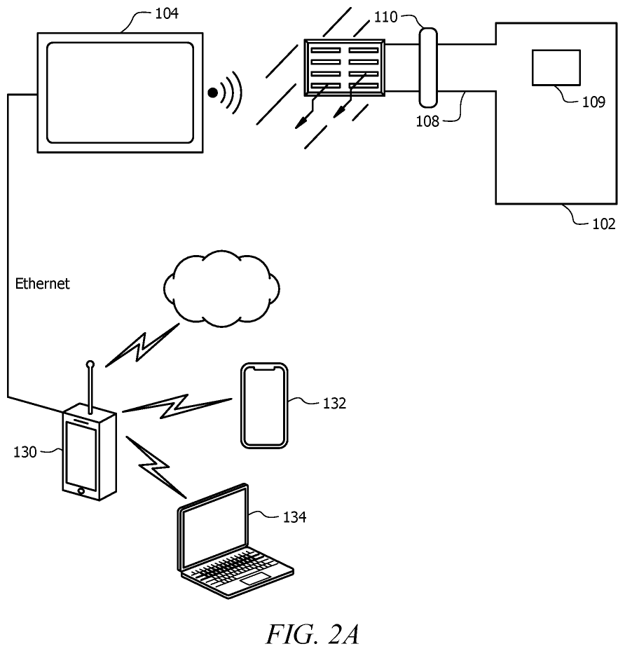

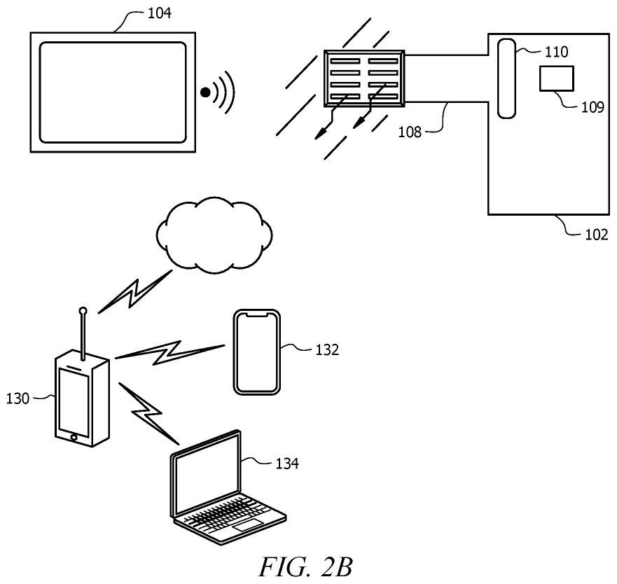

FIG. 2A provides a more detailed view of the intelligent thermostat of the presently described system in communication with associated devices and HVAC duct 108 in which UV-C lamp 110 is installed. In FIG. 2A, intelligent thermostat 104 communicates via wired, such as a Ethernet or RS485, or a wireless protocol, such as Bluetooth or Wi-Fi communication with various local network devices. Intelligent thermostat 104 communicates with other devices through router 130 that provides wired or wireless communication with linked communication devices such as a smartphone 132 or laptop computer or tablet 134. Intelligent thermostat comprises a transmitter and receiver for such communications with associated devices. Router 130 enables communication by linked devices over a wide area network such as the internet. Router 130 also enables communication between local devices.

Intelligent thermostat 104 can be in wired or wireless communication with lamp 110, the latter employing known or proprietary short range communication protocols. In one embodiment, user commands entered on the display screen of intelligent thermostat 104 cause appropriate signals to be transmitted either via wired channel or wireless channel to lamp 110. The following specific control features of the intelligent thermostat 104 provide optimal efficiency in operation of the UV-C lamp.

Control of UV-C lamp 110 by intelligent thermostat 104 enables maximizing effective lamp life via proper control. Intelligent thermostat 104 includes “occupied” and “unoccupied” settings as part of the normal scheduling of the HVAC equipment and triggers an auxiliary relay on the thermostat to turn on/off the UV-C lamp and HVAC fan only when the relevant space is “occupied”. In this example, effective lamp lifetime is increased substantially. Also, for effective air cleaning the appropriate lamp on-time needed, based on the HVAC system airflow (CFM), can be calculated, and used to ensure a minimum run time. In similar fashion, optimal BPI appliance on time is also calculated based on HVAC system air flow to arrive at a minimum BPI appliance run time. Intelligent thermostat 104 in one embodiment includes an intelligent recovery (also known as adaptive recovery or adaptive start) to precondition the room to be at the desired temperature at the scheduled time instead of starting the temperature conditioning at the scheduled time. In this case, the Intelligent thermostat turns on the UV-C lamp and HVAC fan to start the cleaning before the room is ‘occupied’ at the scheduled time. Intelligent thermostat 104 in one embodiment receives data regarding the occupied or unoccupied status of room 100 via one or more associated occupancy sensors (122 in FIG. 1). Occupancy sensors 122 provide real time state data regarding whether or not an occupant is in a room or area.

In one embodiment, as shown in FIG. 1, occupancy sensor 122 is connected to but external to intelligent thermostat via wireless or wired communication. In an alternative embodiment, an occupancy sensor is embedded within intelligent thermostat 104. Occupancy sensor 122 is an optional feature for control of UV-C lamp or BPI appliance based on occupancy in an area that is cooled or heated by the associated HVAC unit. Control according to occupancy sensor 122 data supplements control of the UV-C lamp or BPI appliance according to the scheduled HVAC run times set and stored in intelligent thermostat 104. Upon receipt of an unoccupied status signal, as indicated by a lack of motion, intelligent thermostat controls lamp 110 as discussed below. It is contemplated that operation of the UV-C light or BPI appliance upon receipt of occupancy sensor data from the occupancy sensor causes the UV-C light or BPI appliance to operate when those devices otherwise would not be running according to the thermostat's HVAC run schedule.

As discussed above, in another embodiment a BPI appliance having similar air particle cleansing capability as a UV-C lamp is placed within HVAC system equipment as described in FIG. 2A. That is, in lieu of a UV-C lamp, unit 150 is a BPI appliance that with respect to FIG. 2A is placed within HVAC duct 108 and in close proximity to HVAC vents through which purified air flows back into the room. In this embodiment, the BPI appliance is controlled in the same manner and capacity by the intelligent thermostat 104 embedded controller as described above in connection with FIG. 2A.

FIG. 2B depicts another embodiment and provides a more detailed view of the intelligent thermostat of the presently described system in communication with associated devices and HVAC unit 102 in which UV-C lamp or BPI appliance 110 is installed. The components described in connection with FIG. 2A are the same in this embodiment, except appliance 110 is installed inside the HVAC unit in close proximity to coils 109. Thus, as air particles from the room enter HVAC unit 102 through duct work for air intake into the HVAC unit, appliance 110 treats air particles that attached to and surround coils 109.

Using the scenario above of an eight-hour workday and 221 workdays per year, the effective lamp life is approximately six years. (12,000 hrs./221 days per year/9 hrs. per day). Note that nine hours per day is applied rather than eight hours because the lamp life reduces by one hour each time turned on. The repetitive turning on and off of the BPI appliance also reduces its life cycle. By simply programming the intelligent thermostat to allow the light to run for only an eight-hour period regardless of the HVAC unit itself running for twenty-four hours drastically extends the real lifetime of the appliance.

Another feature enabled by the intelligent thermostat (or remote device connected via wireless or wired communication with the thermostat, or in the standalone device, as the case may be) is proving the lamp(s) or appliance(s) is(are) in operation. Aside from the cost savings realized by extending the life of the lamp or appliance, it is very important from a safety standpoint to establish that the lamp is actually functioning during periods of use. Using an intelligent thermostat with an auxiliary input, either on the thermostat itself or on a remote communicating bus device connected to the thermostat, the thermostat can take any of several methods to determine if the lamp is operating. These include current flow to the lamp, digital or analog output from the UV-C lamp ballast, or data read from a UV-C ray or BPI ion sensitive sensor placed where it can directly see the lamp or appliance output, or a VOC air sensor, including but not limited to devices which can detect Covid molecules. Continuous monitoring of this information by the intelligent thermostat allows operators to confidently allow individuals to occupy and use the relevant space as intended while the destruction of harmful microbes continues.

The intelligent thermostat also indicates when the lamp needs to be replaced. Intelligent thermostat 104 includes an embedded counter enabling lamp use levels to be confirmed. The counter records the number of on-off operations and/or keeps a rolling time of use since last light change. In addition, the current flow measurement is recorded and compared to previous current level samples. A change or difference above a predetermined threshold indicates a degradation in the lamp or appliance's operation, indicating that lamp replacement may be imminent. Similarly, measurement of digital or analog output from the UV-C lamp ballast or BPI appliance and any change thereto can be monitored and if a threshold level of change is met, intelligent thermometer 104 provides an alert that lamp replacement is required. Also, UV-C sensitive sensor located in close proximity to the UV-C lamp is continuously monitored for an interruption or temporary downtime in the UV-C lamp emitting light or that UV-C lamp is not emitting at the required intensity, even if still functioning, to effectively kill microbes. When intelligent thermostat 104 receives such data from the UV-C sensitive sensor, it alerts of a lamp replacement condition.

In another embodiment, both a UV-C lamp and BPI appliance are employed in the same system in a variety of combinations. That is, a UV-C lamp is installed in the HVAC duct as shown in FIGS. 1A and 2A or in close proximity to coil 109 as shown in FIGS. 1B and 2B, or both, while at the same time a BPI standalone appliance is mounted within the room as shown in FIG. 1C. Similarly, a BPI appliance is installed in the HVAC duct or within the HVAC unit close to coil 109, while at the same time an UV-C lamp is mounted within the room. In another embodiment, different combinations of UV-C lamps and BPI appliances are installed. Regardless of the combination, these cleansing units are under control of the smart thermostat for efficient and effective operation.

The intelligent thermostat then alerts when the UV-C the lamp is not operating as described above when various operational conditions are not met. Using the data described above related to current, digital, or analog measurements or UV-C ray or BPI ion sensitive sensor measurements, intelligent thermostat 104 sends an email, SMS, and/or text and/or illuminates or invokes an iconic notification on the thermostat display to alert, either directly or through another electronic method, to the user(s). Thus, either through direct viewing on the thermostat display itself or through an application program installed and executed on a device such as smartphone 132 or tablet or laptop 134, a user is notified of a UV-C lamp or BPI appliance that has failed or its failure is imminent. Upon receipt of the alert, the responsible party can take corrective action by replacing the lamp or checking proper operation of the associated ballasts or the like.

Intelligent thermostat 104 (or remote device connected to the thermostat remote sensor bus, or a standalone device, as the case may be) also records and/or reports historical lamp operation. In some environments, more than one lamp must be installed in associated with a particular HVAC system. This can be due to the plenum size and cubic feet per minute (cfm) of air flow through the HVAC system. The intelligent thermostat includes data collection and storage means, either locally or remotely or both, to store historical information with time/date stamp information of various events. These events include each lamp “on and off” operation and various data described above in connection with current flow to the UV-C lamp or BPI appliance, digital or analog output from the UV-C lamp ballast and UV-C light or BPI ion sensitive sensor data. Some or all of this data can be extracted and included in stored data and included in notification messages and reports or lamp operation. Extracted data can be leveraged by and included in other reporting systems or included in sent email, SMS, and/or text or iconic notification on the thermostat display to provide historical information, either directly or through another electronic method, to relevant users.

Intelligent thermostat 104, through its user interface, provides control features for simple selection of different UV-C lamp and BPI appliance control profiles for different building use cases. The intelligent thermostat's software includes a variety of lamp profiles that allows the user the ability to select among several operation profiles. The user can select the profile most suitable for the room, building or area of interest. In the alternative, the user can create a custom operation profile suited to the particular occupancy and use variables of the particular location. The user created profile can be stored and implemented in the thermostat, in a control device located on the thermostat's remote communication bus, or as an independent control device.

While the disclosed embodiments have been described with reference to one or more particular implementations, these implementations are not intended to limit or restrict the scope or applicability of the invention. Those having ordinary skill in the art will recognize that many modifications and alterations to the disclosed embodiments are available. Therefore, each of the foregoing embodiments and obvious variants thereof is contemplated as falling within the spirit and scope of the disclosed inventions.

While the invention has been particularly shown and described with reference to a preferred embodiment, it will be understood by those skilled in the art that various changes in form and detail can be made therein without departing from the spirit and scope of the invention.

What is claimed is:

1. A sanitizing system, comprising:
a heating, ventilation and cooling (HVAC) unit;
a thermostat having a processor and executable code which controls operation of the HVAC unit;
a controller associated with the thermostat which controls operation of the an air purification unit; and
storage associated with the thermostat,
wherein a predefined run schedule for the HVAC system is stored in storage, and
wherein the controller operation of the air purification unit is based on the predefined run schedule.

2. The sanitizing system of claim 1, further comprising the air purification unit, wherein the air purification unit consists of one of a group of an ultraviolet germicidal irradiation light and a bipolar ionization appliance.

3. The sanitizing system of claim 1, further comprising
an occupancy detector in communication with the controller,
wherein the controller causes the air purification unit to operate when the controller receives a signal from the occupancy detector indicating an area heated or cooled by the HVAC system contains an occupant.

4. The sanitizing system of claim 1, wherein the controller causes the HVAC to operate according to the predefined run schedule.

5. The sanitizing system of claim 2, wherein the air purification unit is located within an HVAC duct of a structure.

6. The sanitizing system of claim 2, wherein the air purification unit is located within the HVAC system in proximity to the evaporator coil.

7. The sanitizing system of claim 2, wherein the air purification unit is located remotely from the HVAC system as a standalone unit having a first opening for air intake and a second opening for air output.

8. The sanitizing system of claim 2, wherein the controller comprises executable code for determining an optimal run time of the air purification unit.

9. The sanitizing system of claim 1, wherein the controller is external to the thermostat.

10. The sanitizing system of claim 3, wherein the occupancy detector is external to the thermostat.

11. A method for sanitizing air and surfaces in an enclosed area, comprising the steps of:
setting for a thermostat a schedule for a heating, ventilation and cooling (HVAC) system to operate to heat or cool an enclosed area;
controlling operation of an air purification unit according to the schedule for the HVAC system to operate to heat or cool the enclosed area; and
receiving at the thermostat air purification unit data indicating operational parameters of the air purification unit; and
modifying activation from the thermostat of the air purification unit according to the received operational parameters of the air purification unit.

12. The method of claim 11, further comprising,
receiving at the thermostat air purification unit data indicating operational parameters of the air purification unit; and
modifying activation from the thermostat of the air purification unit according to the received operational parameters of the air purification unit.

13. The method of claim 12, further comprising:
receiving sensor data at the thermostat from an occupancy sensor comprising an indication that the enclosed area is occupied by a person; and
activating from the thermostat the air purification unit in response to sensor data received that enclosed area is occupied by a person.

14. The method of claim 11, wherein the air purification unit consists of one of a group of an ultraviolet germicidal irradiation light and a bipolar ionization appliance.

15. A thermostat, comprising:
a controller comprising a processor and executable code for operation of an associated heating, ventilation and cooling (HVAC) system and an air purification unit; and
a memory on which a schedule for operation of the HVAC system is stored, wherein the controller causes operation of the air purification unit according to the stored schedule for operation of the HVAC system.

16. The thermostat of claim 15, further comprising a receiver for receiving a signal from an occupancy sensor indicating whether an area is occupied by a person and data from an air purification unit of a state of the air purification unit.

17. The thermostat of claim 15, wherein the air purification unit consists of one of a group of an ultraviolet germicidal irradiation light and a bipolar ionization appliance.

18. The thermostat of claim 15, wherein the transmitter of the thermostat and the receiver of the thermostat communicate with the air purification unit via a wired or wireless communication schema.

19. The thermostat of claim 15, wherein the control causes operation of the air purification unit according to received data from the air purification unit of the state of the air purification unit.

20. An air purification appliance controller, comprising:
comprising a processor and executable code for controlling operation of an air purification unit;
a receiver for receiving a signal from a remote device of a real time or predetermined schedule for operation of a heating, ventilation and cooling (HVAC) system and data from the air purification unit of a state of the air purification unit;
a memory on which a schedule for operation of the HVAC system is stored; and
a transmitter for sending a control signal to the air purification unit to turn on the air purification unit according to communication from the remote device of the real time or predetermined schedule for operation of the HVAC system.

21. The air purification appliance controller of claim 21, further comprising an air purification unit.

22. The air purification appliance controller of claim 21,
wherein the receiver receives a signal from an occupancy sensor indicating whether an area is occupied by a person and data from an air purification unit of a state of the air purification unit, and
wherein the controller causes the air purification unit to run according to the signal received from the occupancy sensor.

23. The air purification appliance controller of claim 21, wherein the air purification appliance controller of claim 21, further comprising the air purification unit, and wherein the air purification unit consists of one of a group of an ultraviolet germicidal irradiation light and a bipolar ionization appliance.
